(19) United States
(12) Patent Application Publication
(10) Pub. No.: US 2003/0119030 A1
(43) Pub. Date: Jun. 26, 2003

(54) Immunoassay diagnostic probe and a method for use thereof

(76) Inventor: Gil Zilber, Ramat Gan (IL)

Correspondence Address: LOWE HAUPTMAN GILMAN AND BERNER, LLP, 1700 DIAGONAL ROAD, SUITE 300 /310, ALEXANDRIA, VA, 22314, US

(21) Appl. No.: 10/221,665

(22) PCT Filed: Mar. 8, 2001
(86) PCT No.: PCT/IL01/00215

(30) Foreign Application Priority Data
Mar. 15, 2000 (IL) 135076

(51) Int. Cl. C12Q 1/68

(52) U.S. Cl. 435/6

(57) ABSTRACT

A disposable probe, an immunoassay diagnostic device and a method for the quantitative detection of specific biomolecules, being the analyte, in a fluid sample, by any immunoassay procedure based on spectroscopic detection, the disposable probe comprises at least one photodiode having a layer of first immunoreactant molecules attached permanently to its surface, wherein the first immunoreactant binds specifically to the analyte, and the photodiode has electronic connectors for transferring the electronic signal generated in the photodiode, upon exposure to light, to a signal processing unit.

The immunoassay diagnostic device comprises a disposable probe, a non-disposable probe-base and means for electronically connecting the probe to the probe-base, and the probe-base comprises means for reading the signal generated by the disposable probe, a signal processing unit and a display unit. The method for the detection of specific biomolecules in a fluid sample, in particular a serum, by using a disposable probe, comprises the steps of any known immunoassay procedure suitable for the detection of the specific biomolecules, the layer attached to the photodiode surface consists of the first immunoreactant of the immunoassay.

### FIELD OF THE INVENTION

The present invention relates to an immunoassay diagnostic probe and device for the quantitative detection of specific biomolecules. More specifically, the present invention relates to a small and inexpensive disposable probe for the detection of specific biomolecules in immunodiagnostic assays. The disposable probe is comprised of a photodiode with first immuno-reactant (complementary to the specific biomolecules of interest) bound to its surface, optionally packaged together with a signal-processing unit in a single semiconductor chip. Said probe is capable of detecting directly photons emitted by molecules bound to its surface, thus eliminates the need for costly, time-consuming, and complexed technologies commonly used for detection in bioassays by combining together probe and detector, (and optionally signal processor), into one component.

### BACKGROUND OF THE INVENTION

The use of light-producing or color-producing chemical reactions for quantitative detection of proteins, antibodies, DNA and other biomolecules, by immunoassay techniques is widely known. A variety of techniques, including chemiluminescence, fluorescence, and certain enzymatic reactions may be used as detection methods. One of the most commonly used techniques is the ELISA (Enzyme-Linked Immunosorbent Assay).

All of these immunoassay techniques rely on the high specificity of the antigen-antibody binding reaction. In a solid-state immunoassay a specific antigen (or antibody) is attached to a solid support, the sample to be tested is then added and any antibody (or antigen) molecules complementary to that specific antigen, that are present in the sample bind to the antigen. Another tagged component, which binds specifically to the antibody of interest, is then added. The tagging (or labeling) of this third component, whose amount is proportional to the amount of the tested biomolecule can be done by various ways and serves as the means for detection. Other variations of immunoassay involve a fourth component, for example in ELISA procedure, the substrate/chromogens that produce color in the reaction which is catalyzed by the enzyme, or the reagents that undergo chemiluminescent reaction catalyzed by the enzyme, in a chemiluminescence assay.

The use of light-generating reactions for quantitative detection in ligand-binder assays is a rapidly growing field. Automated immunoassay analyzers from several different manufacturers are in commercial use and still others are in development. The technical demand of these assay markets requires detection technologies that are highly sensitive, not overly prone to interference, robust and simple to use. Chemiluminescent processes are well suited for these applications. One reason accounting for the growing popularity of chemiluminescent assays is their exquisite detection sensitivity. Unlike fluorescent measurements, in chemiluminescence assays, there is little or no stimulating background light and the measurement is directly proportional to concentration, and not to the difference between two numbers as in optical density measurements. Another advantage is that in chemiluminescent assays there is no need for a light source. The lack of inherent background and the ability to easily measure very low and very high light intensities with simple instrumentation provide a large potential dynamic range of measurement. The chemiluminescence measurement is therefore relatively simple, requiring only a photo-sensor or a photo-multiplier and the associated electronics to convert, process and record signals.

There are many commercially developed automated immunoassay analyzers that measure the chemiluminescence or fluorescence produced in a bioassay. However, all of these technologies involve relatively large, expensive apparatus that is practical only for a laboratory setting. There is a need for a smaller and more efficient tool for conducting immunoassays outside of the laboratory, such as in a doctor's clinic, patient's home or field application, which would provide immediate and reliable results, and eliminate the need for sending samples to labs for processing. The need also exists for a device that is both simple to use and whose detection sensitivity spans a large wavelength of light intensities.

It is therefore the primary object of the present invention to provide a small, low-cost, and highly sensitive immunoassay detecting unit, which will allow for the direct quantification of specific biomolecules through the use a disposable probe.

It is also the object of the present invention to provide a highly sensitive, accurate, easy to use and easy to maintain device for immunological assays in doctors clinics, at home or at the field, in which the production of chemiluminescene or fluorescence is directly and simply coupled to the production of an electrical current which can be easily interpreted in terms of the analyte concentration.

In the context of the present invention, the terms analyte and target molecules are used interchangeably to describe the specific biomolecules of interest i.e., the molecules to be detected.

The terms probe molecules, first immunoreactant or complementary molecules are used interchangeably to describe the molecules that are used for recognizing and binding the target molecules through bioselective and biospecific affinity, such as exists between the following binding pairs: antibody-antigen, antibody-antibody, protein-receptor, substrate-enzyme, complementary nucleic acid strands, binding protein-nucleic acid, etc., and which is referred sometimes in more general terms as ligand-binder couple.

### SUMMARY OF THE INVENTION

The present invention relates to a disposable probe for the quantitative detection of specific biomolecules, being the analyte, in a fluid sample, by any known immunoassay procedure which is based on spectroscopic detection. The probe of the present invention comprises at least one photodiode chip having a layer of first immunoreactant molecules attached permanently to its surface, said first immunoreactant binds specifically to said analyte, and said photodiode has electronic connectors for transferring the electronic signal generated in the photodiode, upon exposure to light, to a signal processing unit. According to the present invention, part or all of the signal processing components, such as amplifier, A/D converter, etc., can be packaged together with the photodiode in a single semiconductor chip (a photo-sensor chip).

Said analyte and said first immunoreactant can be the two complementary components of any biospecific binding pair such as enzyme-substrate, enzyme-inhibitor, complementary nucleic acid strands, binding protein-nucleic acid and in particular, the analyte and first immunoreactant are the two complementary components of an antigen-antibody or antibody-antibody binding pair.

The probe of the present invention can be used in a chemiluminescence assay, in a fluorescence assay or enzyme-linked immunosorbent assay wherein the enzyme catalyses a color producing reaction

The surface of the photo-sensor can be coated by a thin layer of any suitable material in order to increase the bonding affinity of the immunoreactant molecules and/or to protect the photo-sensor form the environment. The material can be ceramic material or polymeric material such as polyamide polyethylene, cellulose etc. In an embodiment of the present invention this thin layer can be a removable membrane covered by a layer of the immunoreactant molecules and said membrane can be removed from the surface of the photodiode after use and replaced by a fresh membrane. The surface of the photodiode can be also coated by an optical filter capable of passing only a specific range of wavelengths.

The probe of the present invention can be used for the detection of more than one analyte according to two embodiments. Either the probe comprises an array of photo-sensor chips, each covered with a layer of different immunoreactants, or that the surface of the photo-sensor is divided into areas (pixels), each pixel covered with a layer of different immunoreactants, wherein each immunoreactant is specific to different analyte.

The present invention further relates to an immunoassay diagnostic device comprising the disposable probe of the present invention, a non-disposable probe-base and means for electronically connecting said probe to said probe-base, wherein the probe-base comprises means for reading the signal generated by the disposable probe, a signal processing unit and a display unit. The device can further comprising a light source for use in immunoassays based on fluorescence or optical-density measurements.

In an embodiment of the present invention, said device is in the form of a pipette, wherein the pipette body is the probe-base containing the signal processing unit and the display unit, and wherein said disposable probe is attached to, or being an integral part of, a disposable pipette tip and wherein upon fitting said pipette tip to said pipette body, electronic connection is established between the two, allowing the reading and processing of a signal generated in the probe and displaying the result.

The present invention also relates to the method for the detection of specific biomolecules in a fluid sample, in particular a serum, by using the disposable probe and/or device of the present invention, wherein said method comprises the steps of any known immunoassay procedure suitable for the detection of said specific biomolecules and wherein the layer attached to the photo-sensor surface consists of the first immunoreactant of said immunoassay.

### BRIEF DESCRIPTION OF THE DRAWINGS

FIG. 1: A schematic drawing of a disposable immuno-diagnostic probe according to the present invention.

FIG. 2: The steps of the immunoassay procedure using the probe of the present invention

FIG. 3: A preferred embodiment of a device comprising the probe of the present invention.

FIG. 4: A graph showing the intensity of emitted light in a chemiluminescent reaction versus time, as measured by a photo-sensor chip immersed in a vial containing the reaction solution (experiment 3).

### DETAILED DESCRIPTION OF THE INVENTION

The present invention discloses a new simple and inexpensive apparatus for the quantitative detection of specific biomolecules by a disposable probe. The apparatus of the present invention can be fitted to any known, or yet to be found, immunoassay procedure by using the appropriate molecules involved in said immunoassay, providing that the labeling is detected by optical spectroscopic means.

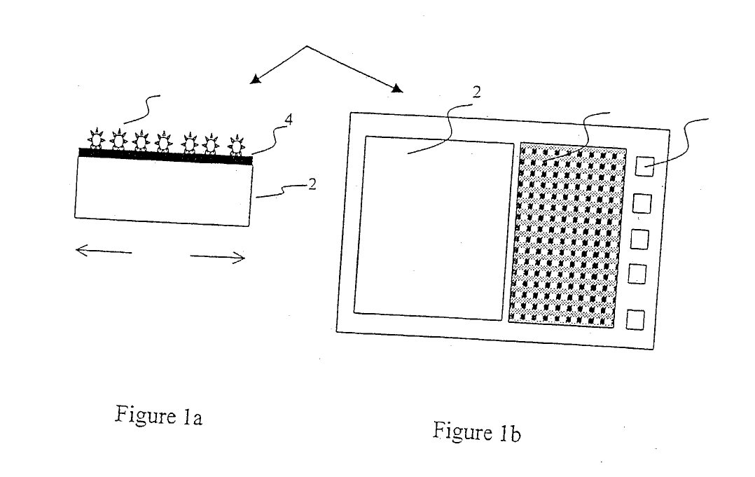

FIG. 1 describes schematically the disposable probe of the present invention. The probe is composed of a layer of probe molecules attached to the surface of a photodiode, wherein the probe molecules are the first immunoreactant of the immunoassay procedure specific to the biomolecules of interest.

The disposable probe optionally includes part or all of the associated signal processing electronic circuits packed together with the photodiode in one semiconductor chip. This semiconductor chip can be produced by any standard semiconductor manufacturing process, such as complementary metal-oxide semiconductor circuitry (CMOS) technology, which assures its low cost and mass production capability, so it may be used only once and then discarded.

It is the common practice in the microelectronic industry to coat the surface of the integrated circuits by various protecting thin layers of different materials such as polyamide, polyethylene, silicone-nitride, silicone-dioxide etc. According to the present invention, the surface of the photo-sensor can be further coated by a thin layer of any material that is suitable for increasing the bonding affinity of the immunoreactant molecules to the photo-sensor. This material can be ceramic material such as silicon dioxide or polymeric material such as cellulose, polyamide polyethylene, etc. According to one embodiment of the present invention this thin layer can be a removable membrane, such as a cellulose membrane, covered by a layer of the immunoreactant molecules and said membrane can be removed from the surface of the photodiode after use and replaced by a fresh unused membrane.

The surface of the photodiode can also be coated by a thin optical filter capable of passing only a specific range of wavelengths.

When the probe is immersed in a serum containing the analyte, the analyte molecules bind to the photo-sensor surface through the probe molecules. After a wash step to remove unbound serum components, the probe is soaked in a solution, which contains a second labeled immunreactant that binds specifically to the analyte. This second reactant can later participate in a chemiluminescence reaction (in a chemiluminescent assay) upon the addition of suitable reagents or can emit light after being exposed to a suitable light source (in a fluorescent assay). The probe can then be inserted into a diagnostic box, or a probe-base, containing a light source (in case of fluorescence) and electronic processing unit. The fluorescence or chemiluminescence from the labeled probe is directly detected by the photodiode and converted into a concentration reading.

The close distance between the light emitting reagent and the detection component (photodiode) assures highly sensitive and reliable results without the need for complex optical assemblies containing lenses, optical scanners and photomultipliers alignment mechanisms which occupy large volume.

The present invention utilizes either the method of chemiluminescence or fluorescence in order to tag the target biomolecule within the bioassay and may be used in conjunction with any number of optical immunoassays that are conventionally used to screen for the presence and amount of specific antibodies in a serum.

Because the light emission and the light detection are in direct coupling within the same probe unit, it is possible to obtain signal reading in a faster and more reliable manner than that of other commonly used methods, which require spectrophotometers or other highly technical equipment. The direct coupling between the photodiode and the chemiluminescent reaction reduces the possibility of error and makes the bioassay more sensitive.

Furthermore, the probe of the present invention is much smaller than the normally used microtiter plates (less than 1 mm² compared to 10 mm²), and therefore only minute volumes of antigen, agents and serum are required, making the device convenient and cost effective. Another advantage, which is due to the small dimension of the probe, is the possibility to make multiple tests on one sample.

According to an embodiment of the present invention the surface of the photodiode is divided into a number of separate areas called pixels (or the probe is comprised from an array of photo-sensor chips) and each pixel (or photodiode chip) is coated by a different antigen. This embodiment provides a way for detecting the presence of several types of antibodies in a given assay.

The present invention offers an accurate and sensitive apparatus to be utilized at doctor's clinic, home and field applications. The use of disposable probe facilitates perfect hygiene and comfort and the simple operating method enables the use of the equipment even by inexperienced persons and can be used for mass screening of very sensitive medical tests.

The versatile probe of the present invention can successfully replace main laboratory devices, and is a valuable tool in the doctor's office and laboratory, small and large, as well as for special applications and secured personalized tests at patients' home. The present probe could be used also at a commercial automated machine after appropriate adjustment.

Preferably, the present invention relates to the detection by a chemiluminescent assay means, of antibodies in a serum, by using a probe, which comprises a photo-sensor chip having the complementary antigen attached to its surface.

While the following detailed description refers specifically to such a preferred embodiment, it does not intend to limit the scope of the invention to immunoassays of this type, but only in order to illustrate the usefulness and efficiency of the invention.

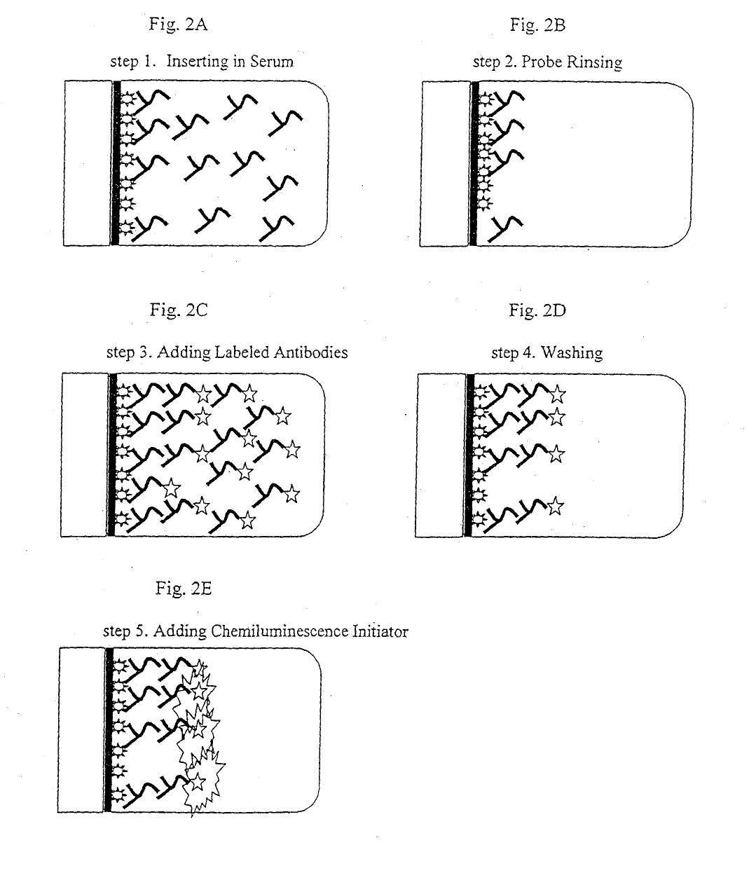

The following detailed description is illustrated sequentially in FIG. 2.

Step 1: The disposable probe, with a specific known antigen bound to its surface, is immersed in a sample which is to be tested for the presence and amount of a specific antibody, said antibody being complementary to the known antigen. The antibodies in the serum bind to the complementary antigen on the probe.

Step 2: After allowing sufficient time for binding, the probe is rinsed to remove unbound serum components.

Step 3: The probe is immersed in a solution of labeled antibodies that bind to the antigen-antibody complex already attached to the probe. The labeling of the antibodies is such that fluorescence or chemiluminescence can be later detected with the use of proper light source or proper reagents, respectively.

Step 4: The probe is washed to remove unbound components.

Step 5: The probe is immersed in an initiator solution that enables a light-emitting reaction only in the presence of the proper labeled antibodies. The emitted light is converted by the photodiode into an electrical current, which can be converted into a concentration reading by any conventionally used signal processing tool.

The present invention further relates to a device for the quantitative detection of specific biomolecules comprising the special probe of the present invention and a probe-base which comprises signal processing unit for processing the signal obtained by the probe and a display unit for displaying the measurements results.

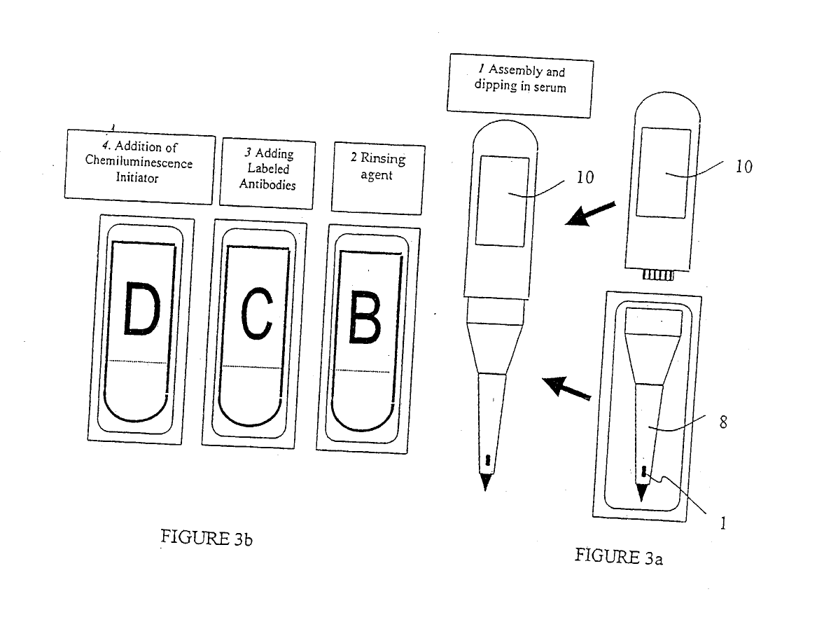

FIG. 3 discloses a preferred embodiment of such a device. According to this preferred embodiment the probe-base is a multiple-use pipette containing a signal processing unit and a display unit (10). The disposable probe of the present invention (1) is attached to a disposable tip (8) fitting the pipette and upon connecting the tip and pipette, electronic connection are established between the two. The pipette is then inserted into a sequence of solutions, according to the immunoassay procedure following the steps of FIG. 2, as shown in FIG. 3b. Alternatively, the solutions might be stored in canisters embedded inside the measuring tip, where a mechanism releases sequentially the agents in pre determined volumes for washing, applying and so on.

This preferred embodiment has many obvious advantages with regard to easiness of operation, test duration and accuracy.

Experimental

The following experiments demonstrate and clarify the present invention and do not mean by any way to limit the scope of the invention.

The experiments are based on the oxidation reaction of luminol by hydrogen peroxide catalyzed by peroxidase, and followed by light emission (luminescence).

The following stock solutions (reagents) were in use:

1. Human IgG, 26 mg/ml.

2. Goat anti-human IgG/HRP conjugate, 3.9 mg/ml of specific IgG.

3. Coating buffer: carbonate buffer, pH 9.6.

4. Blocking solution: 1% BSA in phosphate saline buffer (PBS), pH 7.4.

5. Buffers for washing: PBS, pH 7.4 and PBS containing 0.05% Tween-20.

6. 4-Iodophenol in DMSO, 5 mg/ml.

7. 0.12 mM luminol in 0.1 M Tris-buffer, pH 8.5.

8. Hydrogen peroxide, 4 mM in 0.1 M Tris-buffer, pH 8.5.

9. Horseradish peroxidase (HRP), 0.08 mg/ml in 0.1 M Tris-buffer, pH 8.5.

10. Mixture containing 0.2 ml of reagent 6, 10 ml of reagent 7 and 10 ml of reagent 8.

Experiment 1—Testing the Feasibility of Silicon Photodiode to Adsorb Antigen.

A silicon wafer (1 cm×1 cm) was placed into 3 ml of IgG/HRP conjugate solution in coating buffer at a concentration of 0.02 mg/ml (stock solution 2, diluted 195 times) and incubated for 1 h at 36° C. Then the IgG/HRP solution was poured out, and the silicon wafer was washed with 3 ml of PBS (thrice repeated) and placed into a clean vial. 5 ml of Mixture 10 was added to the vial and the light emission was measured.

Experiment 2—Testing the Feasibility of the ELISA Scheme: 1) Coating a Silicon Wafer with Antibodies 2) Blocking the Unoccupied Sites 3) Adding Labeled Antibodies Against the First Antibodies (Demonstration for Antigen and Labeled Antibodies) 4) Adding Initiator and Viewing the Chemiluminescence Reaction.

A silicon wafer (1 cm×1 cm) was placed into 5 ml of the human IgG solution in coating buffer at a concentration of 0.1 mg/ml (stock solution 1, diluted 260 times) and incubated for 1 h at 36° C. The IgG solution was poured out, and the silicon wafer was washed with 5 ml of PBS (thrice repeated). Then 5 ml of the blocking solution (reagent 4) was added into the vial containing the silicon wafer and incubated for 1 h at 36° C. The blocking solution was poured out, and the silicon wafer was washed 3 times with 5 ml of PBS containing 0.05% Tween-20. At the next stage the washed silicon wafer was incubated for 1 h at 36° C. with 5 ml of anti-human IgG/HRP conjugate in PBS containing 0.05% Tween-20 (concentration of conjugate 0.025 mg/ml relative to specific IgG). After incubation, the IgG/HRP solution was poured out, and silicon wafer was washed 3 times with 5 ml of PBS containing 0.05% Tween-20.

At the last stage 5 ml of Mixture 10 was added, and the light emission was measured.

Experiment 3—Measuring the Emitted Light by Immersed Photodiode During a Chemiluminescence Reaction

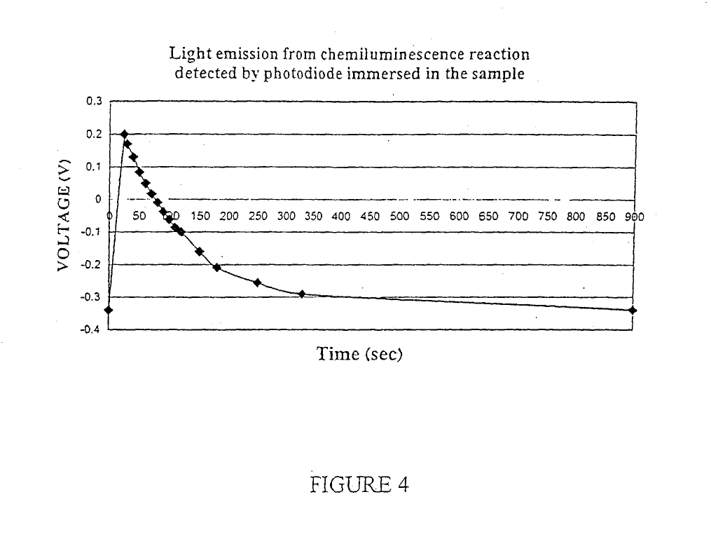

0.1 ml of solution 9 was added to mixture 10. 2.5 ml, from the resulting mixture, was placed into vial (plastic syringe). Then this vial was arranged under a photo-sensor chip, and the emitted light was monitored. FIG. 4 shows the obtained plot of light intensity versus time, as measured by the photo-sensor chip.

Experiment 4—Measuring the Emitted Light From a Chemiluminescence Reaction Activated on the Surface of a Photodiode Coated With a Permanently Attached Antibody Layer.

A commercial photodiode, was placed into 5 ml of the human IgG solution in coating buffer at a concentration of 0.1 mg/ml (stock solution 1, diluted 260 times) and incubated for 1 h at 36° C. The IgG solution was poured out, and the photodiode was washed with 5 ml of PBS (thrice repeated). Then 5 ml of the blocking solution (reagent 4) was added into the vial containing the photodiode and incubated for 1 h at 36° C. The blocking solution was poured out, and the photodiode was washed 3 times with 5 ml of PBS containing 0.05% Tween-20. At the next stage the washed silicon wafer was incubated for 1 h at 36° C. with 5 ml of anti-human IgG/HRP conjugate in PBS containing 0.05% Tween-20 (concentration of conjugate 0.025 mg/ml relative to specific IgG). After incubation, the IgG/HRP solution was poured out, and the photodiode was washed 3 times with 5 ml of PBS containing 0.05% Tween-20.

At the last stage 5 ml of Mixture 10 was added, and the emitted light was monitored. The results was similar to the one described at FIG. 4.

1) A disposable probe for the quantitative detection of specific biomolecules, being the analyte, in a fluid sample, by any immunoassay procedure based on spectroscopic detection, comprising;
at least one photodiode having a layer of first immunoreactant molecules attached permanently to its surface, wherein said first immunoreactant binds specifically to said analyte,
and said photodiode has electronic connectors for transferring the electronic signal generated in the photodiode, upon exposure to light, to a signal processing unit.

2) A disposable immunoassay diagnostic probe according to claim 1 wherein said analyte is one component of an antigen-antibody or antibody-antibody specific reaction and said first immunoassay reactant is the complementary component.

3) A disposable immunoassay diagnostic probe according to claim 1 wherein said analyte and said first immunoreactant are the two components of the binding pair selected from enzyme-substrate, enzyme-inhibitor, complementary nucleic acid strands, binding protein-nucleic acid, or any other biospecific binding pair.

4) A disposable immunoassay diagnostic probe according to claim 1 for use in chemiluminescence assay.

5) A disposable immunoassay diagnostic probe according to claim 1 for use in fluorescence assay.

6) A disposable immunoassay diagnostic probe according to claim 1 for use in enzyme-linked immunosorbent assay wherein the enzyme catalyses a color producing reaction.

7) A disposable immunoassay diagnostic probe according to claim 1 wherein part or all of the signal processing components are packaged together with said photodiode in a single semiconductor chip.

8) A disposable immunoassay diagnostic probe according to claim 1 wherein the surface of the photodiode is coated by an optical filter capable of passing only a specific range of wavelengths.

9) A disposable immunoassay diagnostic probe according to claim 1 wherein the surface of the photodiode is coated by a thin layer of any material suitable for increasing the bonding affinity of said first immunoreactant to the surface of the photodiode and/or to protect the photodiode form the environment.

10) A disposable immunoassay diagnostic probe according to claim 9 wherein said thin layer is a removable membrane covered by a layer of the immunoreactant molecules and said membrane can be removed from the surface of the photodiode after use and replaced by a fresh membrane.

11) A disposable immunoassay diagnostic probe according to claim 1 for the detection of more than one analyte wherein the layer of molecules bound to the photodiode surface comprises more than one reactant, wherein each reactant is bound to a specific defined area of said surface and each react is a first immunoreactant specific to said analyte.

12) A disposable immunoassay diagnostic probe according to claim 1 for the detection of more than one analyte wherein said probe comprises an array of photodiodes, each photodiode covered by a first immunoreactant specific to said analyte.

13) An immunoassay diagnostic device comprising a disposable probe as defined in preceding claims, a non-disposable probe-base and means for electronically connecting said probe to said probe-base, and wherein said probe-base comprises means for reading the signal generated by the disposable probe, a signal processing unit and a display unit.

14) An immunoassay diagnostic device according to claim 13 in the form of a pipette, wherein the pipette body is the probe-base containing the signal processing unit and the display unit, and wherein said disposable probe is attached to, or being an integral part of, a disposable pipette tip and wherein upon fitting said pipette tip to said pipette body, electronic connection is established between the two, allowing the reading and processing of a signal generated in the probe and displaying the result.

15) An immunoassay diagnostic device according to claim 13 further comprising a light source for use in immunoassays based on fluorescence or optical-density measurements.

16) A method for the detection of specific biomolecules in a fluid sample, in particular a serum, by using a disposable probe as defined in claim 1, comprising the steps of any known immunoassay procedure suitable for the detection of said specific biomolecules, wherein the layer attached to the photodiode surface consists of the first immunoreactant of said immunoassay.

17) A method for the detection of specific biomolecules in a fluid sample, in particular a serum, by using an immunoassay diagnostic probe as defined in claim 1 comprising the following steps;
a) immersing said disposable probe in said sample for a predetermined period of time, allowing said tested specific biomolecules, if present in sample, to bind to the probe through binding to the layer of specific first immunoreactant molecules;
b) rinsing said disposable probe to remove unbound molecules;
c) soaking the probe in a solution containing a predetermined concentration of a conjugate that selectively binds to the tested biomolecules, wherein said component is either already labeled by a marker that can be detected by optical means or upon reacting with additional components will produce an optical signal;
d) rinsing said disposable probe to remove unbound molecules;
e) adding, if needed, an appropriate substrate or initiator to produce an optically detectable reaction.
f) exposing the probe to light radiation if needed in order to produce an optically detectable signal.
g) reading the electrical signal generated by said probe in response to said optical signal and interpreting said signal in terms of concentration of the analyte.

18) A method for the detection of specific molecules in a serum according to claim 17 wherein said conjugate of step (c) contains a chemiluminescent molecule that emits light radiation upon addition of a chemiluminescent initiator in step (d).

19) A method for the detection of specific molecules in a serum according to claim 17 wherein said conjugate of step (c) contains a fluorescent molecule that emits light radiation upon exposure to light of a specified wavelength range in step (f).

20) A method for the detection of specific molecules in a serum according to claim 17 wherein said conjugate of step (c) contains an enzyme that catalyzes a reaction that produces a detectable color change upon the addition of an appropriate substrate in step (d) and wherein the detected optical signal is the optical density of the sample measured by exposing the probe to light of a specified wavelength range in step (f).
